SEQUENCE LISTING (20 sequences)

SEQ ID NO: 1 - DNA, 32 bp, Artificial Sequence
  Synthetic oligonucleotide primer for PCR
gaccccttyacaagrtcyatytcyctgagggt

SEQ ID NO: 2 - DNA, 24 bp, Artificial Sequence
  Synthetic oligonucleotide primer for PCR
caragtgtagaaggtcckragrtc

SEQ ID NO: 3 - DNA, 35 bp, Artificial Sequence
  Synthetic oligonucleotide primer for PCR
caagagattgtgtgytaygaragyccamggccaac

SEQ ID NO: 4 - DNA, 22 bp, Artificial Sequence
  Synthetic oligonucleotide primer for PCR
cggagccrcyytccctytgrca

SEQ ID NO: 5 - DNA, 40 bp, Artificial Sequence
  Synthetic oligonucleotide primer for PCR
tataatcacagagaggttaacaatggctgtgagctcaaac

SEQ ID NO: 6 - DNA, 30 bp, Artificial Sequence
  Synthetic oligonucleotide primer for PCR
ctgacgccaccctggtggatacacggtctg

SEQ ID NO: 7 - DNA, 26 bp, Artificial Sequence
  Synthetic oligonucleotide primer for PCR
gaccctaatctcagagaatacttgca

SEQ ID NO: 8 - DNA, 24 bp, Artificial Sequence
  Synthetic oligonucleotide primer for PCR
ccaaaagttaccccagtagttgct

SEQ ID NO: 9 - DNA, 22 bp, Artificial Sequence
  Synthetic oligonucleotide primer for PCR
cttggtgtgaattgcagaatcc

SEQ ID NO: 10 - DNA, 16 bp, Artificial Sequence
  Synthetic oligonucleotide primer for PCR
ggcttcgggcgcaact

SEQ ID NO: 11 - DNA, 3680 bp, Jatropha curcas
acgcggggatgataatacgagtgtagccaacaaaacaaccaataagatatataggtagtggtgcttccgtaatgcctagggatcaatttagagaccctctcgttgttggtcgtgtgattggggatgttttagacccttttacaaaatctatctccctccaggttacttataatcacagagaggttaacaatggctgtgagctcaaaccctctcaagttgtcaaccaacctagggttgatatcggtggagatgatctgaggaccttttatactttggtatttctttttattgttgttgttgttcttcttcttcttcttcttctttcagttgcttttgttttgtttttaaatctaaatgactatacaaacatgggttcttgggttttgaagtgggaattatgattcttttcttcttgaaattgcaggttatggtggaccccgacgcccctagcccaagtgaccctaatctcagagaatacttgcattggtaccttcatattcgatcccatatcatatgattctctcgacaaaattaatgcacacacaattttctttatttctcattcatatatgttcgtatccaatcatagttcaaacaaactttaaatccttcagtatacagatatatatggcttcgatccaatacttgtttcacaaaatcagtggaaaatgtgatttattagcaagttttctttatttatttaacatgccattccagttgaaggtaaattaaattaaatgataaggaagaaagaaagaaaacaaaaaatggaaatggcagagagcagtaggaaggaaaggtaaaaaaagctaagtacggaaaaagagaagtggtagtggcacaaaaacaaaaaaggtgtcgttgtagacaatgttatgctctttggggcgcggggtggtggggtttgaaagagacatatataggtgtcgtgtaatcacatcagtaagtcgttgctttttcaggttagagtaactgagtaagtatagaatatgtcaattttccctttgtctgttttgaaaattcatcgatgcatatttttctttcgagaaagagttcggaccgcacataatttgaagaaagtaggttcggatttactaggtttcttgggtaatcagttgggtcctacgtgtaccatgattctgcttctgggtacactatcttacatcacaacacaattcactaaattctgttctatttcaattttctactgtgggtcctgctttctacagtgattgattggagtaaggtgatccttgcgttgtctctttttacttttacttcattttaaaatggcaaccttttattattattattattattattataatataggtgcttgaggaaaaaaaaaatttcttctttacaaaatattaaccgcacctatcattataaataaaaaaaaatacttatttagatcagataactcaataaaaataaaaattttttaaaaaccattcatatttttttttctcagtaatatcctcttttaataaaaatttatttaattaatggatacattgtgcaccaggtttagataagtcaatctcttataagcaactcttttacgaacaataaatattttttgtaaaatatttgaaaatagtagcaaccacacctattattctgtaaaccagacttatttattttgtaaaccgaacaaatttaaaagataggcatattttacgaaataaattaaaaatattacaaaatataaattcttaaaaaccttacaaagtattagcgactgaaactgttatatattttgttaggcatgaagggttgggggaaccctcggttcaaaaattttaaattatggaattatgaaccggctttttaacggctgattctagttcggtttcaattttgactgattctgatgtggttttgatgaattgactattcctattatagttttgctatgtgtattatttttttttaaagaaaaataatttagttctaaggtgtttttctttcaaaaatattaatgtgaatttaaaaaaaatattgatagattttatctcaactaacttttaaagtgctcgtactcataagaaataaaattaattaattttgcaaaataatcaattaaatcatcattttattttttttataaatgttattaattaaataaattactagaaaattttgaaaaatgcttcaaataatgtaattttgaattaagtaaaaatttaaaaagagaaaatattaaactctcatttgtatcaaacattataattttatactactaaatatttctttttttttttgccattttactttttaatatttttaactttttaagtaaagtgtatttaaaaaaaaaaactaagtaagatgtattatgtagtaaaagattatttagaggtgtataaaataggttaaatgattataattttaaaaataaaaaaaaatttaatttgaaccatattaaaactgccgatttcattcaggatcaaaatataccatttatatgaattttaatcgacttaaactatcaattttaatttaaaattttaaatagacagtttcaatcggtgaacagtttatttactataccgaactgatgcagatgtctatattttgtaatccgataactgcatgaaaaaatacaacaactttttactgacttgtcaagaaatgttagacgatagtaatgagtgctatcattttttaattacatgacatgacatgacatgacttttcttcacaccataatatgttggaccaagctagtcctaccactgtattacgttaggactcagtcagagagatggaataatggaacagaccaaaaaaacaaaacttttgttttaaattataatattcatattcattttgaggaaatcgaaatcgtttttggactcaaaagtgcaattccagtacgtatagtacgctgagaaagatgcaaaaggcgtctttgcagttgaagaacatttttaatatatatagggaagcatcaaccagtgcatgttcatgcagatttttttttttttatttatttaaaaaaaaaagaagatacaggcttaattatgatatgttgaatttatctaaacttgtgcaggttggtgactgatattccagcaactactggggtaacttttggtgagactttaattaattaattaattaattaagttttgtttaattatttagatgtcattttcttgaatctctccgttgagaccaaatcaagaatgtatatatgtgcagggcaagagatagtgtgctatgagagcccgcgaccatcgttggggattcatcggttcgtatttatattgttccggcagctgggaaggcagaccgtgtatccaccagggtggcgtcagaatttcaacactagagatttcgctgagctctacaatcttggttcaccggtggctgctgtttattttaattgccagagggagagtggcaccggaggacggagacggtgattcagttatatattaattactccaatctatacttgattaatcactaatgatcttaagaataagaagaagaagaagatgtattacggtaatgctatgcatggatccatgtgataatatttttatattttattgtggttctgatctctacatatatataattcaaccgaactaaaaagatcacaaatgctatcaaatttactgaatattaatctctaaaaaaaaaaaaaaaaaaaaaaaaaaaaaagt

SEQ ID NO: 12 - DNA, 848 bp, Jatropha curcas
acgcggggatgataatacgagtgtagccaacaaaacaaccaataagatatataggtagtggtgcttccgtaatgcctagggatcaatttagagaccctctcgttgttggtcgtgtgattggggatgttttagacccttttacaaaatctatctccctccaggttacttataatcacagagaggttaacaatggctgtgagctcaaaccctctcaagttgtcaaccaacctagggttgatatcggtggagatgatctgaggaccttttatactttggttatggtggaccccgacgcccctagcccaagtgaccctaatctcagagaatacttgcattggttggtgactgatattccagcaactactggggtaacttttgggcaagagatagtgtgctatgagagcccgcgaccatcgttggggattcatcggttcgtatttatattgttccggcagctgggaaggcagaccgtgtatccaccagggtggcgtcagaatttcaacactagagatttcgctgagctctacaatcttggttcaccggtggctgctgtttattttaattgccagagggagagtggcaccggaggacggagacggtgattcagttatatattaattactccaatctatacttgattaatcactaatgatcttaagaataagaagaagaagaagatgtattacggtaatgctatgcatggatccatgtgataatatttttatattttattgtggttctgatctctacatatatataattcaaccgaactaaaaagatcacaaatgctatcaaatttactgaatattaatctctaaaaaaaaaaaaaaaaaaaaaaaaaaaaaagt

SEQ ID NO: 13 - DNA, 531 bp, Jatropha curcas
atgcctagggatcaatttagagaccctctcgttgttggtcgtgtgattggggatgttttagacccttttacaaaatctatctccctccaggttacttataatcacagagaggttaacaatggctgtgagctcaaaccctctcaagttgtcaaccaacctagggttgatatcggtggagatgatctgaggaccttttatactttggttatggtggaccccgacgcccctagcccaagtgaccctaatctcagagaatacttgcattggttggtgactgatattccagcaactactggggtaacttttgggcaagagatagtgtgctatgagagcccgcgaccatcgttggggattcatcggttcgtatttatattgttccggcagctgggaaggcagaccgtgtatccaccagggtggcgtcagaatttcaacactagagatttcgctgagctctacaatcttggttcaccggtggctgctgtttattttaattgccagagggagagtggcaccggaggacggagacggtga

SEQ ID NO: 14 - protein, 176 aa, Jatropha curcas
MPRDQFRDPLVVGRVIGDVLDPFTKSISLQVTYNHREVNNGCELKPSQVVNQPRVDIGGDDLRTFYTLVMVDPDAPSPSDPNLREYLHWLVTDIPATTGVTFGQEIVCYESPRPSLGIHRFVFILFRQLGRQTVYPPGWRQNFNTRDFAELYNLGSPVAAVYFNCQRESGTGGRRR

SEQ ID NO: 15 - DNA, 37 bp, Artificial Sequence
  Synthetic oligonucleotide primer for PCR
atggatccaacaatgcctagggatcaatttagagacc

SEQ ID NO: 16 - DNA, 30 bp, Artificial Sequence
  Synthetic oligonucleotide primer for PCR
atactagttcaccgtctccgtcctccggtg

SEQ ID NO: 17 - DNA, 25 bp, Artificial Sequence
  Synthetic oligonucleotide primer for PCR
atgcctagggatcaatttagagacc

SEQ ID NO: 18 - DNA, 25 bp, Artificial Sequence
  Synthetic oligonucleotide primer for PCR
agccattgttaacctctctgtgatt

SEQ ID NO: 19 - DNA, 27 bp, Artificial Sequence
  Synthetic oligonucleotide primer for PCR
acgcggggatgataatacgagtgtagc

SEQ ID NO: 20 - DNA, 39 bp, Artificial Sequence
  Synthetic oligonucleotide primer for PCR
agagattaatattcagtaaatttgatagcatttgtgatc

(19) United States
(12) Patent Application Publication
(10) Pub. No.: US 2011/0061125 A1
(43) Pub. Date: Mar. 10, 2011

(54) GENES AND PROTEINS FOR CONTROLLING FLOWERING TIME, AND USE OF THE SAME

(75) Inventors: Takanori SAIJO, Toyonaka-shi (JP); Akitsu Nagasawa, Kobe-shi (JP)

(73) Assignee: SUMITOMO CHEMICAL COMPANY, LIMITED, Tokyo (JP)

(21) Appl. No.: 12/862,527

(22) Filed: Aug. 24, 2010

(30) Foreign Application Priority Data
Aug. 25, 2009 (JP) 2009-194379
Mar. 11, 2010 (JP) 2010-054992

(51) Int. Cl. C12N 15/87; C07H 21/04; C12N 15/63; C07K 14/415; A01H 5/00

(52) U.S. Cl. 800/278; 536/23.6; 536/23.1; 435/320.1; 530/370; 800/298

(57) ABSTRACT

The present invention provides the polypeptide of SEQ ID NO: 14, a polynucleotide encoding the polypeptide, a method for controlling the flowering time of a plant which comprises introducing the polynucleotide into a plant, and so on.

### BACKGROUND OF THE INVENTION

1. Field of the Invention

The present invention relates to flowering time control genes derived from plants, particularly from plants of the order Euphorbiales, such as Jatropha curcas, proteins encoded thereby and use of the genes and proteins.

2. Description of the Related Art

In recent years, various plants draw attention as a source of biofuels, as well as food. A tremendous amount of such plants are required to support a vast number of humans; however, the area of farmland is limited. Thus, studies have been made to achieve the maximum yield from the limited area of farmland. Recently, an attempt has been made to shorten the lifecycle of plants by artificially controlling the flowering time and the like, thereby increasing plant yield.

In general, the flowering time of plants is determined also by exogenous factors, such as day length, temperature and nutritional states, as well as endogenous factors peculiar to each plant variety. Traditionally, the flowering time of plants has been controlled by modulating exogenous factors. However, special facilities, for example, light illumination systems and/or temperature regulating systems, are required in order to modulate such exogenous factors, and it causes a problem of high costs, including costs of illumination and/or air-conditioning, as well as high facility cost.

Thus, researchers have been recently attempted to identify plant genes involved in control of flowering time (see, for example, Kobayashi Y. et al., 1999, Science, 286, 1960-1962) and to use them to control the flowering time of plants.

For instance, Unexamined Japanese Patent Application Publication No. (hereinafter, JP-A) 2000-139250 (published on May 23, 2000) teaches that genes regulated downstream from a gene regulated directly by day length are explored to find out a gene capable of shortening the flowering time, and that as a result, this gene can be used to greatly reduce the time from seeding to seed harvesting. More specifically, JP 2000-139250-A discloses that flowering is accelerated by forcibly expressing the flowering gene (FT gene: Flowering Locus T gene) in Arabidopsis thaliana.

To date, FT genes have been identified in several plant species. For instance, flowering genes have been identified in A. thaliana, rice, wheat, ryegrass, chenopod, orange, pumpkin, apple, tomato, grape, and poplar species (see, for example, Igarashi T. et al., 2008, Plant Cell Physiol., 49(3), 291-300).

The FT gene expression is increased in the phloem of vascular bundles in response to, for example, day length, and the FT protein that is the FT gene product migrates to shoot apices and interacts with a bZIP transcription factor, called FD, preferentially expressed in shoot apices, thereby accelerating the flowering time (see, for example, Kobayashi Y. and Weigel D., 2007, Genes Dev., 21, 2371-2384).

### SUMMARY OF THE INVENTION

No flowering genes have yet been isolated from plants considered very useful in terms of industry, such as plants of the order Euphorbiales, represented by Jatropha curcas. There is no report of success in controlling the flowering time of these plants by using the flowering genes.

In view of the conventional problems described above, the present inventors have accomplished the present invention. An object of the present invention is to control the flowering time of plants considered industrially very useful, such as plants of the order Euphorbiales, represented by Jatropha curcas.

For model organisms such as A. thaliana, many researchers make efforts to explore them and a plenty of information, regarding them, including gene sequences, is publicly available. However, there are only a small number of researchers who works with J. curcas, and information regarding this plant, including its gene sequences, is still limited. In addition, the species distance is large between A. thaliana and J. curcas, and homology of the gene sequences has been considered low. Thus, the present inventors attempted to isolate the FT gene by preparing a plurality of degenerate primers.

At first, it was unclear at what developmental stage, in what portion, under what weather conditions, and in what amount the FT gene is expressed in J. curcas, and the cloning of the J. curcas FT gene using cDNA as template was found to be unsuccessful. Therefore, the inventors initially used genomic DNA as template to obtain partial fragments of the J. curcas FT gene, and then conducted expression analysis of this gene, based on the partial fragments obtained. As a result, the inventors have identified at what leaf position and under what weather conditions the J. curcas FT gene is highly expressed.

As a result of a large number of trial and error experiments, the inventors have succeeded in isolating the FT gene from J. curcas, thereby completing the present invention.

Specifically, the present invention encompasses the following inventions:

[1] A polynucleotide encoding a polypeptide selected from the group consisting of:

(a) the polypeptide of SEQ ID NO: 14;

(b) a polypeptide of a variant of SEQ ID NO: 14, wherein the variant is SEQ ID NO: 14 having one or several substituted, deleted, inserted or added amino acid residues, and wherein the polypeptide has a function of regulating the flowering time of a plant;

(c) a polypeptide having a homology of 90% or more with the polypeptide of SEQ ID NO: 14, wherein the polypeptide has a function of regulating the flowering time of a plant; and

(d) a polypeptide of a variant of SEQ ID NO: 14, wherein the variant is SEQ ID NO: 14 having one conservatively substituted amino acid residue, and wherein the polypeptide has a function of regulating the flowering time of a plant.

[2] A polynucleotide selected from the group consisting of:

(e) the polynucleotide of SEQ ID NO: 11, 12 or 13;

(f) the polynucleotide encoding the antisense sequence of the polynucleotide of SEQ ID NO: 11, 12 or 13;

(g) a polynucleotide comprising a polynucleotide amplified with a set of primers of SEQ ID NOs: 17 and 18;

(h) a polynucleotide comprising a polynucleotide amplified with a set of primers of SEQ ID NOs: 19 and 20;

(i) a polynucleotide having a function of regulating the flowering time of a plant, wherein the polynucleotide is a variant of a polynucleotide selected from the group consisting of the polynucleotides (e) to (h) having one to 30 substituted, deleted, inserted or added nucleotides;

(j) a polynucleotide having a function of regulating the flowering time of a plant and hybridizing under a stringent condition to a polynucleotide consisting of a nucleotide sequence complementary to a polynucleotide selected from the group consisting of the polynucleotides (e) to (h); and

(k) a polyucleotide having a function of regulating the flowering time of a plant and having a homology of 84% or more with a polyucleotide selected from the group consisting of the polynucleotides (e) to (h).

[3] The polynucleotide of [1], wherein the polynucleotide is derived from a plant of the subclass Rosidae.

[4] A recombinant vector comprising any of the polynucleotides of [1] to [3].

[5] The recombinant vector of [4], wherein the vector is a plasmid vector, viral vector, phage vector, or cosmid vector.

[6] A polypeptide selected from the group consisting of:

(a) the polypeptide of SEQ ID NO: 14;

(b) a polypeptide of a variant of SEQ ID NO: 14, wherein the variant is SEQ ID NO: 14 having one or several substituted, deleted, inserted or added amino acid residues, and wherein the polypeptide has a function of regulating the flowering time of a plant;

(c) a polypeptide having a homology of 90% or more with the polypeptide of SEQ ID NO: 14, which polypeptide has a function of regulating the flowering time of a plant; and

(d) a polypeptide of a variant of SEQ ID NO: 14, wherein the variant is SEQ ID NO: 14 having one conservatively substituted amino acid residue, and wherein the polypeptide has a function of regulating the flowering time of a plant.

[7] A method for controlling the flowering time of a plant which comprises introducing the polynucleotide of any of [1] to [3], the recombinant vector of [4] or [5], or the polypeptide of [6] into a plant.

[8] A method for producing a transgenic plant which comprises introducing the polynucleotide of any of [1] to [3], the recombinant vector of [4] or [5], or the polypeptide of [6] into a plant.

[9] A transgenic plant into which the polynucleotide of any of [1] to [3], the recombinant vector of [4] or [5], or the polypeptide of [6] has been introduced.

The present invention has an effect of controlling, such as accelerating or delaying, the flowering time of plants considered industrially useful, such as Jatropha curcas of the order Euphorbiales.

### BRIEF DESCRIPTION OF THE DRAWINGS

FIG. 1 is a graph showing the results of quantification of mRNA from JcFT gene by using real-time PCR in Examples.

FIG. 2 is a graph showing the results of quantification of mRNA from JcFT gene by using real-time PCR in Examples.

FIG. 3 shows a photograph of a transgenic A. thaliana plant into which pRI-35S-To71sGFP-CR has been introduced in Examples.

FIG. 4 shows a photograph of a transgenic rice plant into which pRH-2×35S-faiJcFT-CR has been introduced in Examples (arrowhead indicates a flower bud).

### DETAILED DESCRIPTION OF THE PREFERRED EMBODIMENTS

Embodiments of the present invention are illustrated in detail below, but they are not to be construed as limiting the present invention. All the non-patent and patent documents are herein incorporated by reference in their entirety.

As used herein, the term “polypeptide” is used exchangeably with “peptide” or “protein”. As used herein, the term “polynucleotide” is used exchangeably with “gene”, “nucleic acid”, or “nucleic acid molecule”, which is intended to mean “a nucleotide polymer”. As used herein, the term “nucleotide sequence” is used exchangeably with “nucleic acid sequence” or “base sequence”, which is represented by a sequence of deoxyribonucleotides (abbreviated as A, G, C, and T).

1. Polynucleotides

In the embodiments of the present invention, the polynucleotide may be a polynucleotide encoding a polypeptide selected from the group consisting of:

(a) the polypeptide of SEQ ID NO: 14;

(b) a polypeptide of a variant of SEQ ID NO: 14, wherein the variant is SEQ ID NO: 14 having one or several amino acid residues substituted, deleted, inserted or added, and wherein the polypeptide has a function of regulating the flowering time of a plant;

(c) a polypeptide having a homology of 90% or more with the polypeptide of SEQ ID NO: 14, which polypeptide has a function of regulating the flowering time of a plant; and

(d) a polypeptide of a variant of SEQ ID NO: 14, wherein the variant is SEQ ID NO: 14 having one conservatively substituted amino acid residue, and wherein the polypeptide has a function of regulating the flowering time of a plant.

The polypeptide of SEQ ID NO: 14 refers to a protein (hereinafter, sometimes referred to as JcFT protein) encoded by a FT gene (hereinafter, sometimes referred to as JcFT gene) identified by the present inventors, which is derived from Jatropha curcas.

In the embodiments of the present invention, the polynucleotide may be any of the polynucleotide selected from the group consisting of:

(e) a polynucleotide of SEQ ID NO: 11, 12 or 13;

(f) a polynucleotide encoding the antisense sequence of the polynucleotide of SEQ ID NO: 11, 12 or 13;

(g) a polynucleotide containing a polynucleotide amplified with a set of primers of SEQ ID NOs: 17 and 18;

(h) a polynucleotide containing a polynucleotide amplified with a set of primers of SEQ ID NOs: 19 and 20;

(i) a polynucleotide having a function of regulating the flowering time of a plant, wherein the polynucleotide is a variant of a polynucleotide selected from the group consisting of the polynucleotides (e) to (h) having one to 30 nucleotides substituted, deleted, inserted or added;

(j) a polynucleotide having a function of regulating the flowering time of a plant and hybridizing under a stringent condition to a polynucleotide consisting of a nucleotide sequence complementary to a polynucleotide selected from the group consisting of the polynucleotides (e) to (h); and

(k) a polyucleotide having a function of regulating the flowering time of a plant and having a homology of 84% or more with a polyucleotide selected from the group consisting of the polynucleotides (e) to (h).

The polynucleotides (g) and (h) contain a polynucleotide amplified with each primer set; however, there is no particular limitation on a nucleotide sequence part other than that amplified with each primer set. The polynucleotides (g) and (h) may consist of solely a polynucleotide amplified with each primer set.

The polynucleotide of SEQ ID NO: 11 is genomic DNA of the JcFT gene; the polynucleotide of SEQ ID NO: 12 is full-length cDNA of the JcFT gene; and the polynucleotide of SEQ ID NO: 13 is DNA of translated region of the JcFT gene.

The primer set of SEQ ID NOs: 17 and 18 is a primer set that is able to amplify a part of the nucleotide sequence corresponding to the translated region of the JcFT gene, specifically the nucleotide sequence of nucleotides 1-124 of SEQ ID NO: 13; and the primer set of SEQ ID NOs: 19 and 20 is a primer set that is able to amplify a part of the full-length cDNA of the JcFT gene, specifically the nucleotide sequence of nucleotides 1-816 of SEQ ID NO: 12. The specific sequences of the primers are shown below:

(SEQ ID NO: 17)
JcFTL-2F:
5′-ATGCCTAGGGATCAATTTAGAGACC-3′;
(SEQ ID NO: 18)
JcFTL-2RC:
5′-AGCCATTGTTAACCTCTCTGTGATT-3′;
(SEQ ID NO: 19)
JcFTL-3F:
5′-ACGCGGGGATGATAATACGAGTGTAGC-3′;
(SEQ ID NO: 20)
JcFTL-3RC:
5′-AGAGATTAATATTCAGTAAATTTGATAGCATTTGTGATC-3′.

The nucleotide sequences of four primers described above have low homology with the sequences of other plant FT genes. Therefore, these primers may be used to discriminate the J. curcas FT gene from other plant FT genes more distinctly. The polynucleotides per se amplified with these primer sets may be used to discriminate the J. curcas FT gene from other plant FT genes.

The polypeptide encoded by the polynucleotide amplified with the primer set of SEQ ID NOs: 19 and 20, per se, has a function of accelerating the flowering time of plants.

In the embodiments of the present invention, the polynucleotides may be used to modulate the amount and timing of expression of the FT gene in plants, and as a result, the flowering time of plants can be controlled. By way of an example, the polynucleotides of this embodiment which correspond to the sense strand of the FT gene may be used to accomplish the increase in the expression level of the FT gene and/or the acceleration of the timing of expression in plants, thereby accelerating the flowering time of plants. Further, the polynucleotides of this embodiment which correspond to the antisense strand of the FT gene may be used to accomplish the decrease in the expression level of the FT gene and/or the delay of the timing of expression in plants, thereby delaying the flowering time of plants. The section below entitled “4. Method for Controlling Flowering Time of Plants” discloses the details of the method for controlling the flowering time of plants.

In the embodiments of the present invention, the polynucleotides may be in the form of DNA such as cDNA or genomic DNA, or in the form of RNA such as mRNA. DNA or RNA may be a double strand or single strand. A single-stranded DNA or RNA may be a coding strand (sense strand) or non-coding strand (antisense strand).

In the embodiments of the present invention, the polynucleotides may be chemically synthesized or may be modified in the codon usage so that the expression of the encoded protein can be improved. In the embodiments of the present invention, the polynucleotides may be isolated from nature.

When a polynucleotide isolated from nature is used as the polynucleotide of the embodiments, examples of the origin of the polynucleotide preferably includes, but not particularly limited to, a plant of the subclass Rosidae, a plant of the order Euphorbiales, a plant of the family Euphorbiaceae, or Jatropha curcas; among these plants, a plant of the subclass Rosidae is preferable, a plant of the order Euphorbiales is more preferable, a plant of the family Euphorbiaceae is even more preferable, and a Jatropha curcas plant is most preferable.

In the embodiments of the present invention, the polynucleotide may be one encoding a polypeptide of a variant of SEQ ID NO: 14, which variant is SEQ ID NO: 14 having one or several amino acid residues substituted, deleted, inserted and/or added. The site at which one or several amino acid residues are substituted, deleted, inserted and/or added may be any site in the amino acid sequence, as long as the polypeptide with one or several amino acid residues substituted, deleted, inserted and/or added has the function of regulating the flowering time of a plant. As used herein, the term “one or several amino acid residues” refers specifically to up to 10 amino acid residues in number, preferably to up to 6 amino acid residues, more preferably to up to 2 amino acid residues and even more preferably to one amino acid residue.

When the amino acids are mutated, for example, by substitution, it is preferable to be conservatively substituted. This means that a particular amino acid residue is substituted with a different amino acid in which the properties of the amino acid side-chain are conserved. Non-limited examples of such the conservative substitution include substitution between hydrophobic amino acids such as alanine, isoleucine, leucine, methionine, phenylalanine, proline, tryptophan, tyrosine and valine, substitution between hydrophilic amino acids such as arginine, aspartic acid, asparagine, cysteine, glutamic acid, glutamine, glycine, histidine, lysine, serine and threonine, substitution between amino acids having an aliphatic side chain such as glycine, alanine, valine, leucine, isoleucine and proline, substitution between amino acids having a hydroxy-containing side chain such as serine, threonine and tyrosine, substitution between amino acids having a sulfur atom-containing side chain such as cysteine and methionine, substitution between amino acids having a carboxylic acid- and amide-containing side chain such as aspartic acid, asparagine, glutamic acid and glutamine, substitution between amino acids having a base-containing side chain such as arginine, lysine and histidine, and substitution between amino acids having an aromatic-containing side chain such as histidine, phenylalanine, tyrosine and tryptophan. The substitutions between amino acids having the same amino acid side-chain properties may retain the biological activity of the polypeptide.

In the embodiments of the present invention, the polynucleotide may be a variant of a polynucleotide selected from the group consisting of the polynucleotides (e) to (h), which variant has one to 30 nucleotides substituted, deleted, inserted and/or added. The site at which nucleotides are substituted, deleted, inserted and/or added may be any site, as long as the polynucleotide with substituted, deleted, inserted and/or added nucleotides has the function of regulating the flowering time of a plant.

A polynucleotide as described above may be obtained by substitution, deletion, insertion, and/or addition of one or more nucleotides of a particular polynucleotide. Examples of specific methods for altering nucleotides include methods using a commercially available kit (e.g. Transformer Site-Directed Mutagenesis Kit: Clonetech; QuickChange Site Directed Mutagenesis Kit: Stratagene) and methods using polymerase chain reaction (PCR). These methods are well known to those skilled in the art.

In the embodiments of the present invention, the polynucleotide may be a polynucleotide hybridizing, under a stringent condition, to a polynucleotide consisting of a nucleotide sequence complementary to a polynucleotide selected from the group consisting of the polynucleotides (e) to (h).

As used herein, the term “stringent conditions” refers to conditions that permit hybridization at a temperature 15° C. below, preferably 10° C. below the thermal melting temperature (Tm value) of nucleic acids having a high homology with each other, for example, a completely matched hybrid. As a specific example, hybridization is conducted at 68° C. for 20 hours in a conventional hybridization buffer.

More specifically, the “stringent conditions” refer to those that allow formation of a nucleotide-sequence-specific double-stranded polynucleotide and do not allow formation of a non-specific double-stranded polynucleotide. In other words, the conditions refer to those that permit hybridization of nucleic acids having a high homology with each other at a temperature 15° C. below, preferably 10° C. below, or more preferably 5° C. below the thermal melting temperature (Tm value) of, for example, a completely matched hybrid. For example, the conditions refer to those that permit hybridization at 68° C. for 20 hours in a conventional hybridization buffer. More specifically, the hybridization is conducted for 16 to 24 hours at 60 to 68° C., preferably at 65° C., more preferably at 68° C. in a buffer containing 0.9 MNaCl, 0.09M sodium citrate, 0.01 M sodium phosphate (pH 6.8), 1 mM EDTA (pH 8.0), 0.5% SDS, and 1×Denhardt's solution, followed by washing twice for 15 minutes under the condition of 60 to 68° C., preferably 65° C., more preferably 68° C. with a buffer containing 0.3 M NaCl, 0.03 M sodium citrate, and 1% SDS. Those skilled in the art would readily be able to conduct hybridization under such a condition by reference to, for example, Molecular Cloning (Sambrook J. et al., Molecular Cloning: a Laboratory Manual 2nd ed., Cold Spring Harbor Laboratory Press, 10 Skyline Drive Plainview, N.Y. (1989)).

In the embodiments of the present invention, the polynucleotide may be one encoding a polypeptide having a homology of 90% or more with the polypeptide of SEQ ID NO: 14, which polypeptide has a function of regulating the flowering time of a plant. It would be more preferable that the homology with the polypeptide of SEQ ID NO: 14 is 95% or more, and even more preferable that the homology with the polypeptide of SEQ ID NO: 14 is 98% or more. Further, in the embodiments of the present invention, the polynucleotide may be a polyucleotide having a homology of 84% or more with a polyucleotide selected from among the above-defined polynucleotides (e) to (h), which polynucleotide has a function of regulating the flowering time of a plant. It would be more preferable that the homology with a polypeptide selected from among the above-defined polynucleotides (e) to (h) is 90% or more. According to the construction, the flowering time of various plants may be suitably controlled. The flowering time of plants, inter alia, preferably the flowering time of a plant of the subclass Rosidae, more preferably that of the order Euphorbiales, even more preferably that of the family Euphorbiaceae, and most preferably that of Jatropha curcas may be controlled.

In the present invention, the term “homology” is intended to mean the homology between two nucleotide sequences or two amino acid sequences. The homology is determined by comparing two sequences aligned under optimal conditions over the sequences to be compared. In order to make an optimal alignment of nucleotide or amino acid sequences to be compared, additions or deletions (for example, gaps) may be permitted. Such a sequence homology can be calculated using a program, such as FASTA (Pearson & Lipman, 1988, PNAS, 4: 2444-2448 (1988), BLAST (Altschul et al., 1990, Journal of Molecular Biology, 215: 403-410, and CLUSTAL W (Thompson et al., 1994, Nucleic Acid Research, 22: 4673-4680).

The foregoing programs are publicly available from the WEB pages of the International DNA Databank managed by DNA Data Bank of Japan (at Center for Information Biology and DNA Data Bank of Japan: CIB/DDBJ). Further, sequence homology can be calculated using commercially available sequence analysis softwares. Specifically, sequence homology can be calculated by aligning sequences by performing homology analysis using the Smith-Waterman program in DNASYS Pro ver. 2.06 (Hitachi Software Engineering).

In the embodiments of the present invention, while the polynucleotide may consist of solely the polynucleotide encoding the polypeptide of the present invention described below, it may contain additional nucleotide sequences. Examples of the nucleotide sequences to be added include, but not limited to, nucleotide sequences that encode labels (e.g. histidine tag, Myc tag, and FLAG tag), nucleotide sequences encoding polypeptides that can form a fusion protein with the polypeptide of the present invention (e.g. GST and MBP), promoter sequences (e.g. plant-derived, yeast-derived, pharge-derived, and E. coli-derived promoter sequences), and nucleotide sequences encoding signal-sequences (e.g. endoplasmic reticulum transport signals and secretion sequences). Examples of the positions at which these nucleotide sequences are added include, but not particularly limited to, the 5′- and 3′-ends of the polynucleotides.

2. Recombinant Vectors

In the embodiments of the present invention, the recombinant vectors contain the polynucleotide of the present invention described above.

The vectors of the embodiments may be used to modulate the expression level of the FT gene in the plant, and as a result, the flowering time of the plant can be controlled.

There is no particular limitation on the recombinant vectors of the embodiments. Preferably they are plasmid vectors, viral vectors, phage vectors, and cosmid vectors. Among these vectors, plasmid or viral vectors are more preferable. According to the above-defined construction, a desired host may be trasfected.

While there is no particular limitation on the more specific constructs of the recombinant vectors of the embodiments, vectors containing an expression control sequence are preferable. The term “expression control sequence” is intended to mean a nucleotide sequence for controlling the expression of a desired gene. While there is no particular limitation on expression control sequences, they may include, for example, promoter sequences, enhancer sequences, terminator sequences, 5′-untranslated region sequences, 3′-untranslated region sequences, initiation codon sequences, intron splicing signal sequences, translational frame maintenance sequences, and termination codon sequences. The recombinant vectors of the embodiments may contain only one of these specific expression-control sequences, or may contain more than one of them. When the vectors contain more than one expression control sequences, there is no particular limitation on the combination of the sequences.

The “promoter sequence” described above is intended to mean a minimum nucleotide sequence sufficient to initiate the transcription of a gene. The promoter sequences used include, but not limited to, constitutive promoters, tissue-specific promoters, and inducible promoters which induce transcription in response to particular stimulations. It is preferable to select a suitable promoter, depending on the intended use of the recombinant vector.

There is no particular limitation on the constitutive promoters described above. It is preferable to use, for example, CaMV 35S promoter (Benfey P. N. & Chua N. H., 1990, Science 250: 959-966), PG10-90 (JP 09-131187-A), ubiquitin promoters (WO 01/094394), and actin promoters (WO 00/070067).

There is no particular limitation on the tissue-specific promoters described above. It is preferable to use, for example, soybean seed glycinin promoter (JP 06-189777-A), prolamine promoter (WO 2004/056993), kidney bean seed phaseolin promoter (WO 91/013993), rapeseed napin promoter (WO 91/013972), Arabidopsis thaliana Sultr2;2 promoter (Takahasi H. et al., 2000, Plant J. 23: 171-82), and Agrobacterium rolC promoter (Almon E. et al., 1997, Physiol. 115: 1599-1607).

There is no particular limitation on the inducible promoters described above. It is preferable to use, for example, copper ion inducible promoter (WO 08/111,661), steroid hormone inducible promoter (U.S. Pat. No. 6,063,985), ethanol inducible system (WO 93/21334), tetracycline-inducible system (Weinmann P. et al., 1994, Plant J., 5: 559-569), herbicide Safener-inducible promoter (Hershey et al., 1991, Plant Mol. Biol., 17: 679), heat-shock-inducible promoter (U.S. Pat. No. 5,447,858), cold-inducible promoter (U.S. Pat. No. 5,847,102) and promoter induced in response to attack by a plant pathogen (U.S. Pat. No. 5,942,662).

The “terminator sequence” described above is intended to mean a minimum sequence sufficient to terminate gene transcription and to add a polyadenine sequence for stabilization of mRNA. There is no particular limitation on the terminator sequences described above. It is preferable to use, for example, NOS terminator, CR16 terminator (JP 2000-166577-A), and soybean seed glycinin terminator (JP 06-189777-A).

3. Polypeptides

In the embodiments of the present invention, the polypeptide may be a polypeptide selected from selected from the group consisting of:

(a) the polypeptide of SEQ ID NO: 14;

(b) a polypeptide of a variant of SEQ ID NO: 14, wherein the variant is SEQ ID NO: 14 having one or several amino acid residues substituted, deleted, inserted or added, and wherein the polypeptide has a function of regulating the flowering time of a plant;

(c) a polypeptide having a homology of 90% or more with the polypeptide of SEQ ID NO: 14, which polypeptide has a function of regulating the flowering time of a plant; and

(d) a polypeptide of a variant of SEQ ID NO: 14, wherein the variant is SEQ ID NO: 14 having one conservatively substituted amino acid residue, and wherein the polypeptide has a function of regulating the flowering time of a plant. The polypeptide of SEQ ID NO: 14 refers to the protein (JcFT protein) encoded by the FT gene (JcFT gene) identified by the present inventors, which is derived from Jatropha curcas.

In the embodiments of the present invention, the polypeptides may be used to modulate the expression level of the FT gene in plants, and as a result, the flowering time of plants may be controlled.

In the embodiments of the present invention, the polynucleotide may be one encoding a polypeptide of a variant of SEQ ID NO: 14, which variant is SEQ ID NO: 14 having one or several amino acid residues substituted, deleted, inserted and/or added. The site at which one or several amino acid residues are substituted, deleted, inserted and/or added may be any site in the amino acid sequence, as long as the polypeptide with one or several amino acid residues substituted, deleted, inserted and/or added has the function of regulating the flowering time of a plant. As used herein, the term “one or several amino acid residues” refers specifically to up to 10 amino acid residues in number, preferably to up to 6 amino acid residues, more preferably to up to 2 amino acid residues and even more preferably to one amino acid residue.

When the amino acids are mutated, for example, by substitution, it is preferable to be conservatively substituted. This means that a particular amino acid residue is substituted with a different amino acid in which the properties of the amino acid side-chain are conserved. Non-limited examples of such the conservative substitution include substitution between hydrophobic amino acids such as alanine, isoleucine, leucine, methionine, phenylalanine, proline, tryptophan, tyrosine and valine, substitution between hydrophilic amino acids such as arginine, aspartic acid, asparagine, cysteine, glutamic acid, glutamine, glycine, histidine, lysine, serine and threonine, substitution between amino acids having an aliphatic side chain such as glycine, alanine, valine, leucine, isoleucine and proline, substitution between amino acids having a hydroxy-containing side chain such as serine, threonine and tyrosine, substitution between amino acids having a sulfur atom-containing side chain such as cysteine and methionine, substitution between amino acids having a carboxylic acid- and amide-containing side chain such as aspartic acid, asparagine, glutamic acid and glutamine, substitution between amino acids having a base-containing side chain such as arginine, lysine and histidine, and substitution between amino acids having an aromatic-containing side chain such as histidine, phenylalanine, tyrosine and tryptophan. The substitutions between amino acids having the same amino acid side-chain properties may retain the biological activity of the polypeptide.

In the embodiments of the present invention, the polypeptides may polypeptides which have a homology of 90% or more with the polypeptide of SEQ ID NO: 14 and have a function of regulating the flowering time of a plant. More preferably, the homology with the polypeptide of SEQ ID NO: 14 may be 95% or more, and even more preferably, 98% or more.

In the embodiments of the present invention, the polypeptide may be produced using an organism which produces the polypeptide and was isolated from nature, or using genetic engineering techniques, or may be chemically synthesized using, for example, an amino acid synthesizer.

Examples of the recombinant protein expression systems preferably used in the genetic engineering techniques include, but not limited to, E. coli expression systems, yeast expression systems, insect cell expression systems, mammalian expression systems, and cell-free expression systems.

In the embodiments of the present invention, examples of the polypeptides include, but not particularly limited to, those modulated by intermolecular and/or intramolecular cross-linking (e.g. disulfide bond), those subjected to chemical modification (e.g., addition of sugar chain, phosphate or other functional groups), those labeled (e.g. with histidine tag), and those connected with a polypeptides that can form a fusion protein with the polypeptide of the present invention (e.g. streptavidin, cytochrome, and GFP). Furthermore, in the embodiments of the present invention, examples of the polypeptides include chimeric proteins constructed by combining a number of fragments, as long as they substantially have the function of regulating the flowering time of a plant.

4. Method for Controlling Flowering Time of Plants

In the embodiments of the present invention, the method for controlling the flowering time of plants includes the steps of introducing the polynucleotide, recombinant vector or polypeptide of the present invention into a plant.

The polynucleotide, recombinant vector and polypeptide of the present invention are described elsewhere in this specification and they are not detailed here.

There is no particular limitation on the plants described above (that is, plants in which the flowering time is controlled), and they may be monocotyledonous or dicotyledonous plants. Specifically, they are preferably gramineous plants, or plants of the subclass Rosidae, of the order Euphorbiales, of the family Euphorbiaceae, or Jatropha curcas. Among these plants, plants of the subclass Rosidae are preferable, plants of the order Euphorbiales are more preferable, plants of the family Euphorbiaceae are even more preferable, and Jatropha curcas is most preferable.

Examples of the plants may include, but not limited to, soybean, pea, kidney bean, alfalfa, lotus, clover, peanut, sweet pea, walnut, tea, cotton, pepper, cucumber, water melon, pumpkin, melon, radish, rapeseed, canola, beet, lettuce, cabbage, broccoli, cauliflower, arabidopsis, tobacco, eggplant, potato, sweet potato, taro, Jerusalem artichoke, tomato, spinach, asparagus, carrot, flax, sesame, endive, chrysanthemum, geranium, antirrhinum, carnation, pink, periwinkle, bouvardia, gypsophila, gerbera, prairie gentian, tulip, stock, statice, cyclamen, saxifraga, swamp chrysanthemum, violet, rose, cherry, apple, pear, grape, strawberry, Japanese apricot, almond, orange, lemon, banana, mango, papaya, kiwi, coffee, Japanese quince, Satsuki azalea, azalea, poinsettia, cassava, oil palm, coconut, olive, gentian, cosmos, morning-glory, sunflower, ginkgo, Japanese cedar, Japanese cypress, poplar, pine, sequoia, oak, willow, eucalyptus, kenaf, water lily, Eucommia, beech, castor oil plant, bamboo, sugar cane, rice, wheat, barley, rye, oat, maize, sorghum, lawn grass, tall fescue, switchgrass, Japanese silver grass, green onion, onion, garlic, lily, Tiger lily, orchid, gladiolus and pineapple.

There is no particular limitation on the methods for introducing the polynucleotide, recombinant vector and polypeptide of the present invention into the above-mentioned plants. Any known method, such as the Agrobacterium system, particle gun, electroporation, the calcium phosphate method, injection, and virus vector systems, may be used.

According to the method of the embodiments of the present invention for controlling the flowering time of plants, it is possible to accelerate the flowering time of a plant (that is, promote flowering) or to delay the flowering time of a plant (that is, inhibit flowering). In the following, one example is further described for the specific method for accelerating the flowering time, and another example for the specific method for delaying the flowering time, but they are not to be construed as limiting the present invention.

<4-1> Acceleration of Flowering Time

Acceleration of the flowering time of a plant may be achieved according to the following methods (A) to (C).

(A) Any given expression control sequence and the FT gene are ligated into any given vector such that the FT gene can be expressed in plant cells. The vector is introduced into plant cells to obtain a transgenic plant, in which the flowering is promoted. Any expression control sequence or vector is not necessarily required, as long as the FT gene is expressible in plant cells. Even a transgenic plant is not necessary to be obtained. Depending on the host plant, the FT gene may be introduced into plant cells such that the FT gene can be expressed in the plant cells.

(B) Any given nucleotide sequence is integrated into the genomic DNA of a plant to enhance the expression of the endogenous FT gene. Preferably, examples of such a given nucleotide sequence include an expression control sequence. More specifically, examples of such an expression control sequence include promoter sequences and enhancer sequences. Such an expression control sequence is operably integrated into the genomic DNA of a plant to enhance the expression of the endogenous FT gene in the plant, thereby promoting flowering of the plant.

(C) The protein encoded by the FT gene is introduced into plant cells by injecting the protein into, for example, phloem sap. The protein encoded by the FT gene migrates to shoot apices and axillary buds to promote flowering of the plant. The protein encoded by the FT gene may be produced using a recombinant microorganism or extracted from a wild-type plant or transgenic plant. Alternatively, grafting may be performed using, as a rootstock, a plant (for example, a transgenic plant) accumulating the protein encoded by the FT gene.

<4-2> Delaying of Flowering Time

Delaying of the flowering time of a plant may be achieved according to the following methods (D) to (I).

(D) Any given expression control sequence and the FT gene are ligated into any given vector such that the antisense sequence of the FT gene can be expressed in plant cells. The vector is introduced into plant cells to obtain a transgenic plant, in which the flowering time is delayed. Any expression control sequence or vector is not necessarily required, as long as the antisense sequence of the FT gene is expressible in plant cells. Depending on the host plant, the antisense sequence of the FT gene may be introduced into plant cells such that the antisense sequence of the FT gene can be expressed in the plant cells.

As used herein, the term “antisense sequence” refers to a DNA or RNA molecule complementary to at least a portion of a particular mRNA molecule. In a plant cell, an antisense sequence hybridizes to a corresponding mRNA to form a double-stranded molecule and thereby inhibits the translation of mRNA.

(E) The full length or a segment of the FT gene is integrated into a VIGS (Virus Induced Gene Silencing)-inducing viral vector and, in turn, the vector is introduced into plant cells to inhibit the expression of the endogenous FT gene, thereby delaying the flowering time of the plant.

(F) It is possible to convert the FT gene into a flowering repressor gene by substituting one or more amino acids of the FT gene with other amino acids (Hanzawa et al., 2005, PNAS, 102: 7748-7753). Any given expression control sequence and a flowering repressor gene are ligated into any given vector such that a flowering repressor gene into which a dominant-negative mutation as described above has been introduced can be expressed in plant cells. The vector is introduced into plant cells to obtain a transgenic plant, in which the flowering time is delayed. Any expression control sequence or vector is not necessarily required, as long as the flowering repressor gene is expressible in plant cells. Depending on the host plant, the flowering repressor gene may be introduced into plant cells such that the flowering repressor gene can be expressed in the plant cells.

As used herein, the term “dominant-negative mutation” refers to a mutation that is introduced in a manner that adversely affects the normal function of the wild-type gene, and that dominantly affects the activity of the wild-type protein. Thus, this mutation may overcome the wild-type phenotype and exhibits a negative phenotype.

(G) Any nucleotide sequence is integrated into the genomic DNA of a plant to inhibit the expression of the endogenous FT gene. Such a nucleotide sequence should be integrated into the genomic DNA of a plant in a manner that inhibits the expression of the endogenous FT gene in the plant. Specifically, examples of the integration include, but not limited to, insertion of any nucleic acid sequence into the endogenous FT gene in the plant or into an expression control sequence responsible for the expression of the endogenous FT gene in the plant.

(H) A nucleotide sequence such as a ribozyme or a triplex-forming oligonucleotide is introduced into plant cells. The ribozyme cleaves mRNA of the FT gene and the triplex-forming oligonucleotide inhibits the transcription or translation of the FT gene, thereby delaying the flowering time of the plant.

As used herein, the term “ribozyme” is intended to mean an RNA molecule that is capable of cleaving single-stranded RNA in a specific manner. The nucleotide sequence encoding a ribozyme may be altered by genetic engineering to prepare a molecule that can recognize a specific nucleotide sequence located in an RNA molecule and cleave the nucleotide sequence (Ceeh, et al., 1988, J. Amer. Med. Assn., 260: 3030).

Further, as used herein, the term “triplex-forming oligonucleotide” refers to a molecule that twines around a duplex DNA to form a triplex, thereby arresting the transcription of the gene. A triplex-forming oligonucleotide can be designed to recognize a particular nucleotide sequence (Maher et al., 1991, Antisense Res. and Dev., 1: 227).

(I) The protein encoded by a flowering repressor gene into which a dominant-negative mutation has been introduced is introduced into plant cells by injecting the protein into, for example, phloem sap. The protein encoded by flowering repressor gene migrates to shoot apices and axillary buds to delay the flowering time of the plant. The protein encoded by the flowering repressor gene may be produced using a recombinant microorganism or extracted from a transgenic plant. Alternatively, grafting may be performed using, as a rootstock, a plant (for example, a transgenic plant) accumulating the protein encoded by the flowering repressor gene.

The methods for accelerating the flowering time and for delaying it may be carried out alone separately or they may be combined arbitrarily within a possible range. Specifically, for example, into a plant in which the FT gene has been functionally disrupted by inserting a nucleotide sequence into the endogenous FT gene of the plant, a foreign FT gene (or a flowering repressor gene) may be newly introduced such that the foreign gene can be expressed in cells of the plant. An additional FT gene (or flowering repressor gene) may be introduced into a plant into which an FT gene (or flowering repressor gene) has been introduced.

In particular, in order to control the flowering time of Jatropha curcas, it is preferable that any of various inducible promoters, for example, a copper ion inducible promoter (e.g. see WO 08/111,661) is ligated to the polynucleotide of the present invention and then introduced into a plant of the order Euphorbiales, in particular, into Jatropha curcas, and that the flowering time of the plant is controlled by exposing the transgenic plant to copper ions. According to this construction, the flowering time of plants can be controlled more effectively as compared with the construction in which, for example, a DNA fragment of the A. thaliana FT gene, which has an amino acid sequence homology of less that 90% with the JcFT protein, is ligated to the above promoter and introduced into a plant. Further, this construct may prevent emerging of undesirable phenotype, such as morphological aberration.

5. Method for Producing Transgenic Plants

In the embodiments of the present invention, the method for producing a transgenic plant includes a step of introducing the polynucleotide, recombinant vector, or polypeptide of the present invention into a plant. More specifically, the method of the embodiments may be carried out based on substantially the same method as described above for the method of the present invention for controlling the flowering time of plants.

The polynucleotide, recombinant vector and polypeptide of the present invention are described elsewhere in this specification and they are not detailed here.

The specific constitution of the plants and specific procedures for introducing the polynucleotide and the like of the present invention are described above in [4. Method for Controlling Flowering Time of Plants], and they are not detailed here.

6. Transgenic Plants

In the embodiments of the present invention, the transgenic plants are transduced with the polynucleotide, recombinant vector, or polypeptide of the present invention.

The transgenic plants of the embodiments may be produced based on the method described above in [5. Method for Producing Transgenic Plants].

In the transgenic plants of the embodiments, the flowering time is controlled. The control includes, but not limited to, acceleration or delaying of the flowering time.

For example, acceleration of the flowering time allows for reducing the lifecycle of the transgenic plant. Thus, it will be possible to harvest (for example, seeds and fruits of) the plants in a shorter period of time. As a result, cultivar improvement may be accomplished in an efficient manner.

On the contrary, delaying the flowering time can prolong the lifecycle of the transgenic plant. Thus, it will be possible to harvest (for example, seeds and fruits of) the plants after they are sufficiently grown over time. As a result, the marketability of plants, such as leafy vegetables and root vegetables, can be improved.

According to the present invention, it is possible to confer tolerance to more different cultivation environments, seeding time, and cultivation areas, since the flowering time can be accelerated or delayed. As a result, the productivity may be improved.

### EXAMPLES

The invention is further illustrated by the following examples, but is not limited thereto.

### Example 1

### Isolation of Fragments of JcFT Gene

Genomic DNA was prepared from true leaves of Jatropha curcas, using DNeasy Plant mini kit (Qiagen). PCR reaction was performed using the genomic DNA as template and four degenerate primers (FTD-1F, FTD-1RC, FTD-2F, and FTD-2RC):

(SEQ ID NO: 1)

| | |
|---|---|
| FTD-1F: | 5′-GACCCCTTYACAAGRTCYATYTCYCTGAGGGT-3′; |

(SEQ ID NO: 2)

| | |
|---|---|
| FTD-1RC: | 5′-CARAGTGTAGAAGGTCCKRAGRTC-3′; |

(SEQ ID NO: 3)

| | |
|---|---|
| FTD-2F: | 5′-CAAGAGATTGTGTGYTAYGARAGYCCAMGGCCAAC-3′; |
| and | |

(SEQ ID NO: 4)

| | |
|---|---|
| FTD-2RC: | 5′-CGGAGCCRCYYTCCCTYTGRCA-3′. |

Mixed bases are indicated, according to IUB codes, by M (A or C), R (A or G), W (A or T), S(C or G), Y (C or T/U), K (G or T/U), V (A or C or G), H (A or C or T/U), D (A or G or T/U), B (C or G or T/U), and N (A or C or G or T/U).

The PCR product was electrophoresed on an agarose gel, and the amplified fragment obtained was then excised from the gel and purified using MagExtractor (TOYOBO). The purified fragment was cloned into pCR4Blunt-TOPO vector using the ZeroBlunt TOPO PCR Cloning kit (Invitrogen).

This vector was used to transform E. coli DH5α cells, and the colonies selected for drug resistance were picked up. The E. coli cells picked up were cultured in LB medium (0.5% yeast extract, 1.0% Bacto tryptone, and 0.5% NaCl), and the plasmids were prepared using the QIAprep spin miniprep kit (Qiagen).

The nucleotide sequence of the amplified fragment inserted into the plasmids was analyzed using the M13 reverse primer, BigDye terminator v3.1 (ABI), and 3100 Genetic Analyzer (ABI).

As a result, information on the nucleotide sequence (144 bp) located in exon 1 of the JcFT gene was obtained by the PCR reaction using FTD-1F and FTD-1RC; and information on the nucleotide sequence (205 bp) located in exon 4 of the JcFT gene was obtained by the PCR reaction using FTD-2F and FTD-2RC.

Based on the sequence information obtained, two specific primers (JcFTL-1F and JcFTL-1RC) were designed:

(SEQ ID NO: 5)
JcFTL-1F:
5′-TATAATCACAGAGAGGTTAACAATGGCTGTGAGCTCAAAC-3′;
and
(SEQ ID NO: 6)
JcFTL-1RC:
5′-CTGACGCCACCCTGGTGGATACACGGTCTG-3′.

The two specific primers designed (JcFTL-1F and JcFTL-1RC) were used to perform PCR reaction using the genomic DNA as template. The PCR product was cloned as described above and the nucleic acid sequence of the amplified fragment was analyzed. As a result, information on the nucleotide sequences of intron 1 (149 bp), exon 2 (62 bp), intron 2 (2575 bp), exon 3 (41 bp), and intron 3 (108 bp) was obtained.

### Example 2

### Analysis of JcFT Gene Expression in Jatropha curcas

Total RNA was prepared from shoot apices and true leaves of the J. curcas plants grown for 2 to 4 months after being sown on medium soil, using the RNeasy Plant mini kit (Qiagen).

Based on the total RNA thus prepared, cDNA was synthesized using the ReverTra Ace qPCR RT kit (TOYOBO). Quantification of mRNA was conducted by SYBR green-based real-time PCR, using the 7500 Fast Real-time PCR system (Applied Biosystems), with the synthesized cDNA as template.

To quantify the mRNA of the JcFT gene, two specific primers (JcFT-1F and JcFT-1RC) were used. As the internal standard, J. curcas 5.8S rRNA (GenBank Accession Number AM774639) was used. To quantify the mRNA of the 5.8S rRNA gene, two specific primers (Jc5.8-1F and Jc5.8-1RC) were used:

| (SEQ ID NO: 7) | |
|---|---|
| JcFT-1F: | 5′-GACCCTAATCTCAGAATACTTGCA-3′; |
| (SEQ ID NO: 8) | |
| JcFT-1RC: | 5′-CCAAAAGTTACCCCAGTAGTTGCT-3′; |
| (SEQ ID NO: 9) | |
| Jc5.8-1F: | 5′-CTTGGTGTGAATTGCAGAATCC-3′; |
| and | |
| (SEQ ID NO: 10) | |
| Jc5.8-1RC: | 5′-GGCTTCGGGCGCAACCT-3′ |

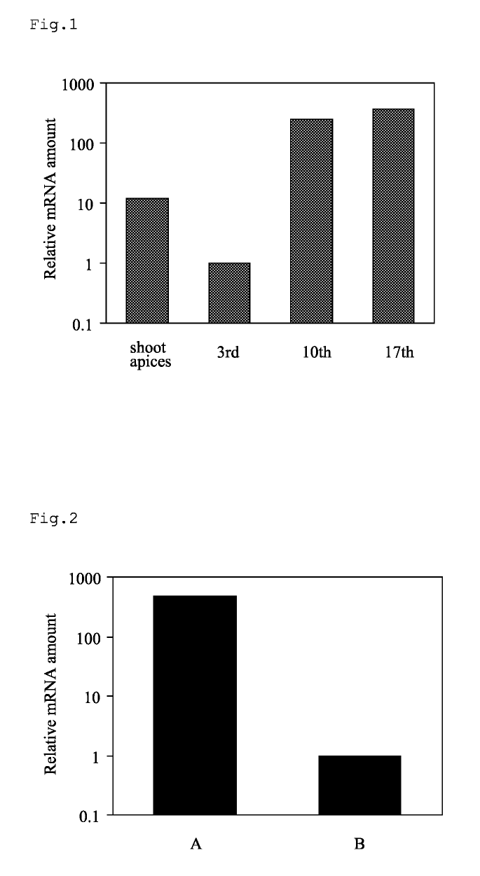

The expression of the JcFT gene in the 10th and 17th true leaves from the top of the J. curcas plants (4 replicate plants) grown under weather conditions A (the average temperature and humidity were 32° C. and 50%, respectively, in the light phase; and the average temperature and humidity were 26° C. and 80%, respectively, in the dark phase with day length 12 hours/night length 12 hours), which are favorable for flower differentiation, was approximately 20-fold higher in average than that in the shoot apices and was approximately 250-fold higher in average than that in the 3rd true leaves from the top (see FIG. 1).

The expression of the JcFT gene in the 10th true leaves from the top of the J. curcas plants (6 replicate plants) grown under weather conditions A (the average temperature and humidity were 32° C. and 50%, respectively, in the light phase; and the average temperature and humidity were 26° C. and 80%, respectively, in the dark phase with day length 12 hours/night length 12 hours), which are favorable for flower differentiation, was approximately 500-fold higher in average than that in the shoot apices and was approximately 250-fold higher in average than that in the 10th true leaves from the top of the J. curcas plants (6 replicate plants) grown under weather conditions B (the average temperature and humidity were 40° C. and 10%, respectively, in the light phase; and the average temperature and humidity were 20° C. and 40%, respectively, in the dark phase with day length 12 hours/night length 12 hours), which are unfavorable for flower differentiation (see FIG. 2). These expression analyses have identified the leaf position and weather conditions that allow high expression of the JcFT gene.

FIG. 1 shows the results of the quantification of the mRNA of the JcFT gene by real-time PCR. In the figure, the mean value of the 4 replicates in the 3rd leaves from the top is represented by 1, and the mean values of the 4 replicates in the shoot apices, 10th leaves, and 17th leaves are respectively indicated by relative values. FIG. 2 also shows the results of the quantification of the mRNA of the JcFT gene by real-time PCR. In the figure, the mean value of the 6 replicates grown under the weather conditions B is represented by 1, and the mean value of the 6 replicates grown under the weather conditions A is indicated by a relative value.

### Example 3

### Isolation of JcFT Gene

Total RNA was prepared from 10th true leaves from the top of the J. curcas plants that were grown under the weather conditions favorable for flower differentiation, using the RNeasy Plant mini kit (Qiagen).

Based on the total RNA thus prepared, the first-strand cDNA for 5′- and 3′-RACE was synthesized using the SMART RACE cDNA Amplification kit (Clonetech) and PrimeScript Reverse Transcriptase (TAKARA BIO).

A specific primer (JcFTL-1RC) and the primer mix (UPM) attached to the SMART RACE cDNA Amplification kit (Clonetech) were used to perform the 5′-RACE reaction using the cDNA synthesized for 5′-RACE as template. A specific primer (JcFTL-1F) and the primer mix (UPM) attached to the SMART RACE cDNA Amplification kit (Clonetech) were used to perform the 3′-RACE reaction using the cDNA synthesized for 3′-RACE as template.

Each RACE reaction product was cloned and sequenced as described above. As a result, information on the nucleotide sequences of the 5′- and 3′-ends of the 5′-untranslated region (71 bp), exon 1 (204 bp), exon 4 (224 bp), and the 3′-untranslated region (246 bp) was obtained.

The information regarding the full-length sequence of the JcFT gene, for instance, the nucleotide sequence in the genomic DNA (see SEQ ID NO: 11), the nucleotide sequence of the full length of the cDNA (see SEQ ID NO: 12), the nucleotide sequence of the translated region (see SEQ ID NO: 13), and the amino acid sequence of the protein (see SEQ ID NO: 14), was thus obtained.

### Example 4

### Construction of Expression Cassette for the JcFT Gene

Plasmid p35S-ACE1/VP16AD-CR (see WO 2008/111661A2) was treated with the restriction enzymes HindIII and EcoRI to obtain an expression cassette for transcription factors from the plasmid. To pRI909 (TAKARA BIO) treated with the restriction enzymes HindIII and EcoRI, the expression cassette for transcription factors was ligated to obtain pRI-35S-ACE1/VP16AD-CR.

Plasmid pMRE4/35S(-46)-To71sGFP (see WO 2008111661A2) was treated with the restriction enzymes XbaI and SacI to obtain the To71sGFP gene from the plasmid. To pRI-35S-ACE1/VP16AD-CR treated with the restriction enzymes XbaI and SacI, the To71sGFP gene was ligated to obtain pRI-35S-To71sGFP-CR.

Total RNA was prepared from true leaves of the J. curcas plants, using the RNeasy Plant mini kit (Qiagen). Based on the total RNA thus prepared, cDNA was synthesized using the ReverTra Ace qPCR RT kit (TOYOBO). PCR reaction was performed using two specific primers (BamJFT-1F and SpeJFT-1RC) with the synthesized cDNA as template:

(SEQ ID NO: 15)
BamJFT-1F:
5′-ATGGATCCAACAATGCCTAGGGATCAATTTAGAGACC -3′;
(SEQ ID NO: 16)
SpeJFT-1RC:
5′-ATACTAGTTCACCGTCTCCGTCCTCCGGTG-3′;

The PCR product was blunted and phosphorylated using the Blunting Kination Ligation kit (TAKARA BIO), and then treated with the restriction enzyme BamHI. Subsequently, the PCR product was ligated into pRI-35S-To71sGFP-CR which had been treated with the restriction enzyme SacI, then blunted and phosphorylated using the Blunting Kination Ligation kit (TAKARA BIO), and treated with the restriction enzyme BamHI. Thus, pRI-35S-To71JcFT-CR was obtained.

### Example 5

### Evaluation of Function of JcFT Gene in Transgenic Arabidopsis thaliana

Plasmids pRI-35S-To71sGFP-CR and pRI-35S-To71JcFT-CR, prepared in Example 4, were introduced into agrobacterium (Agrobacterium tumefaciens strain C58C1). The agrobacterium was cultured on LB-agar medium (0.5% yeast extract, 1.0% Bacto tryptone, 0.5% NaCl, and 1% agar) supplemented with 50 mg/L kanamycin, 100 mg/L ampicillin, and 100 mg/L rifampicin. Drug-resistant colonies were selected to obtain recombinant agrobacterium.

The recombinant agrobacterium thus obtained was transduced into A. thaliana plants (Arabidopsis thaliana ecotype Columbia) by infecting the plants with the recombinant agrobacterium, according to the method described in “Lab Manual for Plant Models” (edited by Iwabuchi, M. et al., 2000, Springer-Verlag Tokyo, ISBN 4-431-70881-2 C3045).

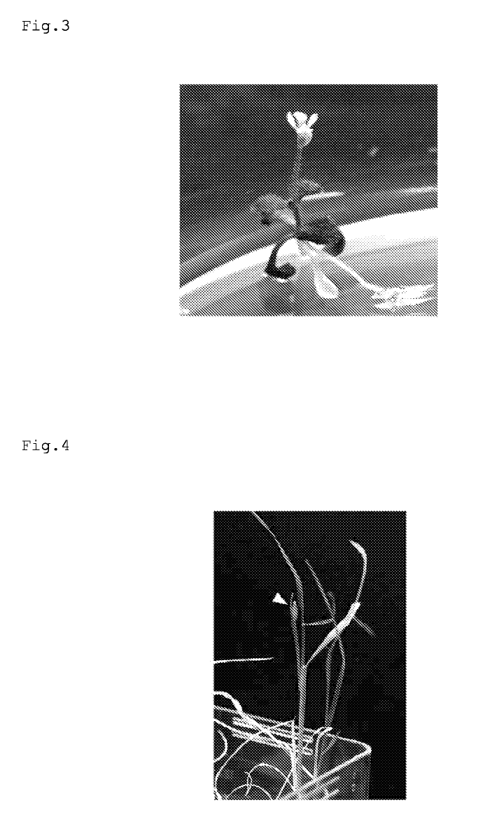

T₁ seeds collected from the transgenic A. thaliana plants were sown and grown on modified MS agar medium (MS minerals, vitamin B₅, 1% sucrose, and 0.8% agar) supplemented with 20 mg/L Benlate, 200 mg/L Claforan, and 25 mg/L kanamycin. The plants were selected for kanamycin resistance. For the transgenic A. thaliana plants transduced with pRI-35S-To71JcFT-CR, flowering was observed on the agar medium on 30 days after sowing (see FIG. 3).

The selected individual plants were transferred to pots filled with medium soil in advance and grown in the biotron to obtain T₂ seeds. The T_{z} seeds thus obtained were sown and grown on modified MS agar medium (MS minerals, vitamin B₅, 2% sucrose, and 0.8% agar) supplemented with 25 mg/L kanamycin. Then, the lines were selected which produced kanamycin resistant plants in a 3:1 ratio at the 5% significance level based on χ² test. The growth conditions for individual plants were as follows: temperature: 23 to 25° C., light phase: 23 hours, and dark phase: 1 hour.

For the selected lines, T₂ seeds were sown on modified MS agar medium (MS minerals, vitamin B₅, 2% sucrose, and 0.8% agar) supplemented with kanamycin at a concentration of 25 mg/L. As a result, flowering of the transgenic A. thaliana plants transduced with pRI-35S-To71JcFT-CR was induced earlier than that of the transgenic A. thaliana plants transduced with pRI-35S-To71sGFP-CR.

On 25 days after sowing, the number of rosette leaves, which is used as an index of flowering time, was counted. As a result, the number of rosette leaves of the transgenic A. thaliana plants transduced with pRI-35S-To71JcFT-CR was 4.3±0.6 (n=24), while the number of rosette leaves of the transgenic A. thaliana plants transduced with pRI-35S-To71sGFP-CR was 5.5±0.7 (n=39). The growth conditions for individual plants were as follows: temperature: 23 to 25° C., light phase: 12 hours, and dark phase: 12 hour.

### Example 6

### Evaluation of the Function of the JcFT Gene in Transgenic Rice Plants

Plasmid pRH909 was constructed in which the kanamycin-resistance NPTII gene of pRI909 (TAKARA BIO) is replaced with the hygromycin-resistance APH4 gene.

Into pRH909, 2×35S promoters (Liu et al. (2002) Plant J. 30: 415-429) and the translation enhancer sequence fai (Mori et al. (2006) Plant Biotech. 23: 55-61), JcFT gene, and CR terminator were inserted to obtain pRH-2×35S-faiJcFTp-CR. Plasmid pRH-2×35S-faiJcFTp-CR thus obtained was introduced into agrobacterium (Agrobacterium tumefaciens strain LBA4404). The agrobacterium was cultured on LB-agar medium (0.5% yeast extract, 1.0% Bacto tryptone, 0.5% NaCl, and 1% agar) supplemented with 50 mg/L kanamycin and 100 mg/L streptomycin. Drug-resistant colonies were selected to obtain recombinant agrobacterium.

The recombinant agrobacterium thus obtained was transduced into rice plants (Oryza sativa subsp. japonica cv. Nipponbare) by infecting the plants with the recombinant agrobacterium, according to the method described in Toki et al. (2006) Plant J. 47: 969-976). Regenerated plants exhibiting hygromycin resistance were obtained. Transgenic rice plants transduced with pRH-2×35S-faiJcFTp-CR formed flower buds earlier (see FIG. 4). The growth conditions for individual plants were as follows: temperature: 23 to 25° C., light phase: 23 hours, and dark phase: 1 hour.

The present invention is not limited to the embodiments described above. Various modifications may be made within the scope of the claims, and other embodiments and examples obtained by appropriately combining the technical means disclosed in different embodiments and examples are encompassed by the technical scope of the present invention.

The present invention may be used for the purpose of producing a plant in which the flowering time is controlled.

What is claimed is:

1. A polynucleotide encoding a polypeptide selected from the group consisting of:
(a) the polypeptide of SEQ ID NO: 14;
(b) a polypeptide of a variant of SEQ ID NO: 14, wherein the variant is SEQ ID NO: 14 having one or several substituted, deleted, inserted or added amino acid residues, and wherein the polypeptide has a function of regulating the flowering time of a plant;
(c) a polypeptide having a homology of 90% or more with the polypeptide of SEQ ID NO: 14, wherein the polypeptide has a function of regulating the flowering time of a plant; and
(d) a polypeptide of a variant of SEQ ID NO: 14, wherein the variant is SEQ ID NO: 14 having one conservatively substituted amino acid residue, and wherein the polypeptide has a function of regulating the flowering time of a plant.

2. A polynucleotide selected from the group consisting of:
(e) the polynucleotide of SEQ ID NO: 11, 12 or 13;
(f) the polynucleotide encoding the antisense sequence of the polynucleotide of SEQ ID NO: 11, 12 or 13;
(g) a polynucleotide comprising a polynucleotide amplified with a set of primers of SEQ ID NOs: 17 and 18;
(h) a polynucleotide comprising a polynucleotide amplified with a set of primers of SEQ ID NOs: 19 and 20;
(i) a polynucleotide having a function of regulating the flowering time of a plant, wherein the polynucleotide is a variant of a polynucleotide selected from the group consisting of the polynucleotides (e) to (h) having one to 30 substituted, deleted, inserted or added nucleotides;
(j) a polynucleotide having a function of regulating the flowering time of a plant and hybridizing under a stringent condition to a polynucleotide consisting of a nucleotide sequence complementary to a polynucleotide selected from the group consisting of the polynucleotides (e) to (h); and
(k) a polyucleotide having a function of regulating the flowering time of a plant and having a homology of 84% or more with a polyucleotide selected from the group consisting of the polynucleotides (e) to (h).

3. The polynucleotide according to claim 1, wherein the polynucleotide is derived from a plant of the subclass Rosidae.

4. A recombinant vector comprising the polynucleotide of claim 1.

5. The recombinant vector according to claim 4, wherein the vector is a plasmid vector, viral vector, phage vector, or cosmid vector.

6. A polypeptide selected from the group consisting of:
(a) the polypeptide of SEQ ID NO: 14;
(b) a polypeptide of a variant of SEQ ID NO: 14, wherein the variant is SEQ ID NO: 14 having one or several substituted, deleted, inserted or added amino acid residues, and wherein the polypeptide has a function of regulating the flowering time of a plant;
(c) a polypeptide having a homology of 90% or more with the polypeptide of SEQ ID NO: 14, which polypeptide has a function of regulating the flowering time of a plant; and
(d) a polypeptide of a variant of SEQ ID NO: 14, wherein the variant is SEQ ID NO: 14 having one conservatively substituted amino acid residue, and wherein the polypeptide has a function of regulating the flowering time of a plant.

7. A method for controlling the flowering time of a plant which comprises introducing the polynucleotide of claim 1 or a recombinant vector comprising the polynucleotide of claim 1 into a plant.

8. A method for controlling the flowering time of a plant which comprises introducing the polypeptide of claim 6 into a plant.

9. A method for producing a transgenic plant which comprises introducing the polynucleotide of claim 1 or a recombinant vector comprising the polynucleotide of claim 1 into a plant.

10. A method for producing a transgenic plant which comprises introducing the polypeptide of claim 6 into a plant.

11. A transgenic plant into which the polynucleotide of claim 1 or a recombinant vector comprising the polynucleotide of claim 1 has been introduced.

12. A transgenic plant into which the polypeptide of claim 6 has been introduced.
